(19) United States
(12) Patent Application Publication
(10) Pub. No.: US 2025/0037874 A1
(43) Pub. Date: Jan. 30, 2025

(54) DEEP LEARNING SUDDEN CARDIAC DEATH SURVIVAL PREDICTION

(71) Applicant: THE JOHNS HOPKINS UNIVERSITY, Baltimore, MD (US)

(72) Inventors: Natalia A. TRAYANOVA, Baltimore, MD (US); Dan POPESCU, Baltimore, MD (US); Mauro MAGGIONI, Baltimore, MD (US); Julie K. SHADE, Baltimore, MD (US)

(21) Appl. No.: 18/715,877

(22) PCT Filed: Dec. 1, 2022
(86) PCT No.: PCT/US2022/051476

§ 371 Date: Jun. 3, 2024

Related U.S. Application Data
(60) Provisional application No. 63/287,395, filed on Dec. 8, 2021.

(51) Int. Cl. G16H 50/30; G16H 50/20

(52) U.S. Cl. CPC G16H 50/30; G16H 50/20

(57) ABSTRACT

Computer-implemented neural network techniques for predicting patient-specific arrhythmic sudden cardiac death survival are presented. The techniques can include obtaining cardiac image data for a patient; obtaining cardiac covariate data for the patient; providing the cardiac image data to a first subnetwork; providing the cardiac covariate data to a second subnetwork; combining an output from the first subnetwork with an output from the second subnetwork to produce survival probability data; and outputting patient-specific arrhythmic sudden cardiac death survival prediction data based on the survival probability data.

### CROSS-REFERENCE TO RELATED APPLICATIONS

This application is the national stage entry of International Patent Application No. PCT/US2022/051476, filed on Dec. 1, 2022, and published as WO 2023/107317 A1 on Jun. 15, 2023, which claims the benefit of U.S. Provisional Patent Application No. 63/287,395, filed on Dec. 8, 2021, which are hereby incorporated by reference herein in their entireties.

### GOVERNMENT FUNDING

This invention was made with government support under grant HL142496 awarded by the National Institutes Health. The government has certain rights in the invention.

### FIELD

This disclosure relates generally to sudden cardiac death survival prediction.

### BACKGROUND

Sudden cardiac death (SCD) continues to be a leading cause of mortality worldwide, with an incidence of 50 to 100 per 100,000 in the general population in Europe and North America, and accounts for 15-20% of all deaths. Patients with coronary artery disease are at the highest risk of SCD due to Arrhythmia (SCDA). While implantable cardioverter devices effectively prevent SCDA, current clinical criteria for implantable cardioverter device candidacy—i.e., left ventricular ejection fraction <30-35%—capture a mere 20% of all SCDA, highlighting the critical need to develop personalized, accurate, and cost-effective arrhythmia risk assessment tools to mitigate this enormous public health and economic burden. Many studies have identified risk factors for SCDA and numerous risk stratification approaches have attempted to transcend the left ventricular ejection fraction criterion. However, limitations in these approaches have been barriers to their clinical implementation.

Prior attempts have broadly stratified populations based on subgroup risk, failing to customize predictions to patients' unique clinical features. SCDA risk has been typically assessed at pre-defined finite time points, ignoring the likely patient-specific time-evolution of the disease. Additionally, in previous work confidence estimates for predictions have been “one-size-fits-all”, varying only by risk subgroup, thus preventing the identification of low-confidence, potentially highly-erroneous prediction outliers. Moreover, few prior studies have validated their results externally or comprehensively compared model performance to standard approaches.

Finally, although arrhythmia arises, mechanistically, from the heterogeneous scar distribution in the disease-remodeled heart, machine learning the features of that distribution has not been adequately explored for risk analysis. Image-derived mechanistic computational models of cardiac electrical function that incorporate scar distribution have proven successful in predicting arrhythmia risk, however, they remain exceedingly computationally intensive and, therefore impractical as a first stage screening tool in a broad population. See Arevalo, H. J. et al., Arrhythmia risk stratification of patients after myocardial infarction using personalized heart models, Nat. communications 7, 1-8 (2016).

### SUMMARY

According to various embodiments, a computer-implemented neural network method of predicting patient-specific arrhythmic sudden cardiac death survival is presented. The method includes: obtaining cardiac image data for a patient; obtaining cardiac covariate data for the patient; providing the cardiac image data to a first subnetwork; providing the cardiac covariate data to a second subnetwork; combining an output from the first subnetwork with an output from the second subnetwork, wherein survival probability data is provided; and outputting arrhythmic sudden cardiac death survival prediction data based on the survival probability data, wherein the arrhythmic sudden cardiac death survival prediction data is specific to the patient.

Various optional features of the above method embodiments include the following. The method may further include treating the patient with an implantable cardioverter device based on the sudden cardiac death survival prediction data. The arrhythmic sudden cardiac death survival prediction data may include an arrhythmic sudden cardiac death survival curve for the patient. The arrhythmic sudden cardiac death survival prediction data may include a probability distribution representing time of predicted arrhythmic sudden cardiac death. The survival probability data may include a probability distribution location datum and a probability distribution scale datum. The method may further include outputting a patient-specific uncertainty assessment for the arrhythmic sudden cardiac death survival prediction data based on the probability distribution scale datum. The combining may include providing the first output and the second output to a third subnetwork that provides the survival probability data. The cardiac covariate data for the patient may include cardiac image measurement data, risk factor data, electrocardiogram data, and medication data for the patient. The cardiac image measurement data may include at least one of left ventricle mass data or infarct size data, the risk factor data may include ejection fraction data, the electrocardiogram data may include at least one of heart rate data or QRS complex data, and the medication data may include at least one of beta blocker medication data, diuretic medication data, digoxin medication data. The first subnetwork may include an encoder-decoder subnetwork, and wherein the second subnetwork comprises a dense subnetwork. The first subnetwork may be trained with training cardiac image data, the training cardiac image data including times until arrhythmic sudden cardiac death events and times until non-arrhythmic-sudden-cardiac-death events, and the second subnetwork may be trained with training cardiac covariate data, the training cardiac covariate data including times until arrhythmic sudden cardiac death events and times until non-arrhythmic-sudden-cardiac-death events.

According to various embodiments, computer-implemented neural network system for predicting patient-specific arrhythmic sudden cardiac death survival is presented. The system includes: a first subnetwork that accepts cardiac image data for a patient and provides corresponding first survival probability data; a second subnetwork that accepts cardiac covariate data for a patient and provides corresponding second survival probability data; and an output that provides arrhythmic sudden cardiac death survival prediction data based on the first survival probability data and the second survival probability data, wherein the arrhythmic sudden cardiac death survival prediction data is specific to the patient.

Various optional features of the above system embodiments include the following. The arrhythmic sudden cardiac death survival prediction data may include an arrhythmic sudden cardiac death survival curve for the patient. The arrhythmic sudden cardiac death survival prediction data may include a probability distribution representing time of predicted arrhythmic sudden cardiac death. The survival probability data may include a probability distribution location datum and a probability distribution scale datum. The output may provide a patient-specific uncertainty assessment for the arrhythmic sudden cardiac death survival prediction data based on the probability distribution scale datum. The system may further include a third subnetwork that combines the first survival probability data and the second survival probability data into third survival probability data, wherein the arrhythmic sudden cardiac death survival prediction data is based on the third survival probability data. The cardiac covariate data for the patient may include cardiac image measurement data, risk factor data, electrocardiogram data, and medication data for the patient. The cardiac image measurement data may include at least one of left ventricle mass data or infarct size data, the risk factor data may include ejection fraction data, the electrocardiogram data may include at least one of heart rate data or QRS complex data, and the medication data may include at least one of beta blocker medication data, diuretic medication data, digoxin medication data. The first subnetwork may include an encoder-decoder subnetwork, and wherein the second subnetwork comprises a dense subnetwork. The first subnetwork may be trained with training cardiac image data, the training cardiac image data comprising times until arrhythmic sudden cardiac death events and times until non-arrhythmic-sudden-cardiac-death events, and the second subnetwork may be trained with training cardiac covariate data, the training cardiac covariate data comprising times until arrhythmic sudden cardiac death events and times until non-arrhythmic-sudden-cardiac-death events.

### DRAWINGS

The above and/or other aspects and advantages will become more apparent and more readily appreciated from the following detailed description of examples, taken in conjunction with the accompanying drawings, in which:

FIG. 1 is a schematic diagram of a system for arrhythmic sudden cardiac death survival prediction according to various embodiments;

FIG. 2 depicts charts illustrating risk prediction performance of a reduction to practice;

FIGS. 3A and 3B depict charts illustrating risk prediction performance of the reduction to practice;

FIG. 4 depicts charts illustrating individual patient survival predictions of the reduction to practice;

FIG. 5 is a schematic diagram of a convolutional subnetwork branch according to various embodiments;

FIG. 6 depicts a graph comparing predictions of the convolutional subnetwork, the dense subnetwork, and entirety of the example reduction to practice to a different representative technique;

FIG. 7 depicts images illustrating image convolutional subnetwork feature prediction according to the example reduction to practice; and

FIG. 8 depicts a chart illustrating dense subnetwork covariate interpretation according to the example reduction to practice.

### DETAILED DESCRIPTION

Embodiments as described herein are described in sufficient detail to enable those skilled in the art to practice the invention and it is to be understood that other embodiments may be utilized and that changes may be made without departing from the scope of the invention. The present description is, therefore, merely exemplary.

Some embodiments provide a robust, generalizable SCDA risk stratifier with the ability to predict individualized, patient-specific risk trajectories. Some embodiments utilize deep learning neural networks and survival analysis to predict patient-specific SCDA survival curves from both raw late gadolinium contrast-enhanced cardiac magnetic resonance images, which visualize heart disease-induced scar distribution, and clinical covariates for patients with ischemic heart disease. The deep-learning-predicted survival curves produced by some embodiments can provide accurate SCDA predictions at all times up to, e.g., ten years in the future.

Some embodiments represent a fundamental change in the approach to arrhythmia risk assessment, as some embodiments directly estimate the uncertainty of their predictions. Such embodiments thus have the potential to significantly shape clinical decision-making regarding arrhythmia risk, offering not a simple “at risk/not at risk” prediction, but instead, an estimate of the time to SCDA together with a quantified sense of how certain the embodiment is about each predicted time to SCDA.

A reduction to practice, discussed throughout this disclosure, was evaluated on multi-center internal validation data, and tested on an external, independent test set, following internal cross-validation. The reduction to practice, which used both raw, unsegmented cardiac images and cardiac covariate data as input, outperformed survival models constructed using either non-imaging or imaging clinical covariates. Brought to clinical practice, this technology has the potential to transform clinical decision-making by offering accurate, generalizable, and interpretable predictions of patient-specific survival probabilities of arrhythmic death over time.

These and other features and advantages are described in detail herein.

### I. Overview

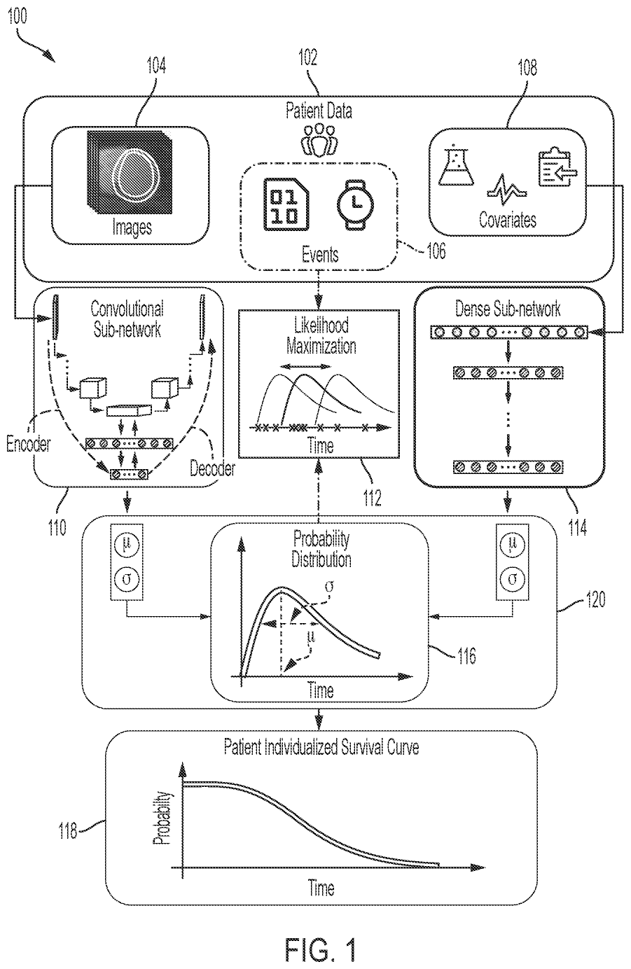

FIG. 1 is a schematic diagram of a system 100 for arrhythmic sudden cardiac death survival prediction according to various embodiments. System 100 includes a deep learning framework that incorporates multiple custom neural subnetworks, including convolutional subnetwork 110 and dense subnetwork 114, that accept different data types, and utilize statistical survival analysis, to predict patient-specific probabilities of SCDA at future time points. As shown in FIG. 1, the subnetworks 110, 114 of system 100 are depicted, by way of non-limiting example, as being included in a left branch and a right branch. Each branch can separately predict patient-specific survival probability data. In the reduction to practice, the survival probability data included two parameters: location (μ) and scale (σ), which fully characterize a log-logistic probability distribution 116 of the patient-specific time to SCDA. The survival probability data (e.g., μ and σ as estimated by each branch and then combined by ensembling subnetwork 120) are then used to generate arrhythmic sudden cardiac death survival prediction data, which may include, by way of non-limiting example, a patient-specific SCDA survival curve, a patient-specific probability distribution representing time of predicted SCDA, and/or a patient-specific probability of SCDA at a particular point in time. Details of the left branch, the right branch, and the training of system 100 are described presently.

The left branch accepts cardiac image data 104. The cardiac image data 104 may be in any of a variety of forms, e.g., static images, dynamic images (e.g., video), or both. In the reduction to practice, and by way of non-limiting example, the cardiac image data 104 included unsegmented late gadolinium contrast-enhanced (LGE) cardiac magnetic resonance (CMR) images that visualized the patients' 3-D ventricle geometry and contrast-enhanced remodeled tissue. In the left branch, the cardiac image data 104 is automatically segmented and used as input by a 3-D encoder-decoder convolutional subnetwork 110. The segmentation may be performed using, e.g., techniques as described in Section VII, to segment the left ventricle myocardium (scar segmentation is not required). The encoder-decoder design of the convolutional subnetwork 110 ensures that resulting imaging features retain sufficient information to be able to reconstruct the original images. The convolutional subnetwork 110 is trained to discover and extract imaging features associated with SCDA risk directly from the cardiac image data 104. Once trained, the left branch provides survival probability data (e.g., location μ and scale σ) for patient-specific input cardiac image data.

The right branch accepts cardiac covariate data 108. The cardiac covariate data 108 may be in any of a variety of forms, e.g., static data (such as numeric values), dynamic data (such as ECG curve data), or both. The 22 cardiac covariates that were used in the reduction to practice are presented in Table 2, in Section V. In the right branch, cardiac covariate data 108, e.g., values for the 22 cardiac covariates in Table 2, are provided to a dense subnetwork 114, which discovers and extracts nonlinear relationships between the input variables. Once trained, the right branch outputs survival probability data (e.g., location μ and scale σ) for patient-specific input cardiac covariate data.

For training, patient data 102, including both the cardiac image data 104 used by the left branch and the cardiac covariate data 108 used by the right branch, are labeled with event data 106, which includes a time to an event and an indicator of the type of event to produce cardiac image training data and cardiac covariate training data. The time to the event in event data 106 is measured relative to the time that the data is collected from the respective patient, e.g., measured in days. The event indicator of the event data may be, by way of non-limiting example, a zero or one, to indicate whether the event was a SCDA of the respective patient or otherwise (e.g., a death of the respective patient from a competing risk or a termination of available data for the respective patient). During training, the neural network weights for subnetworks 110, 114, and 120 are optimized via a maximum likelihood process 112, in which a probability distribution is sought to best match the observed survival data.

The outputs of the subnetworks 110, 114 are combined (e.g., ensembled) by ensembling subnetwork 120 in a way that best fits the observed training data to estimate patient-specific survival probability data (e.g., μ and σ) representing, respectively, the most probable time to SCDA (T_{SCDA}) and a measure of uncertainty in the prediction. These two parameters determine, for each patient, SCDA survival prediction data, e.g., an SCDA-specific survival curve 118, within a given statistical model, e.g., a log-logistic probability distribution 116.

The SCDA survival prediction data may be used as a basis to make clinical decisions regarding the patient. For example, the SCDA survival prediction data may be used to determine whether to implant the patient with an implantable cardioverter device. Such decisions may be based on the T_{SCDA} and/or the uncertainty of the prediction. According to some embodiments, the patient is implanted with an implantable cardioverter device if T_{SCDA} is less than a predetermined time interval, such as by way of non-limiting example, five years or ten years.

### II. Overall Risk Prediction Performance

This section presents performance results of the reduction to practice discussed throughout this disclosure.

The reduction to practice was developed and internally validated using data from 156 patients with ischemic cardiomyopathy (ICM) enrolled in the Left Ventricle Structural Predictors of SCD (LVSPSCD) prospective observational study (see Section VII). The performance of the reduction to practice was evaluated comprehensively on this internal set using Harrell's concordance-index (c-index)—an estimate of the probability of assigning the correct risk order given two randomly selected patients (range is [0, 1], higher is better)—and the integrated Brier score (B̅s̅)—an estimate of the squared difference between predicted survival probability and 0/1 outcome by a certain time, averaged over all times (range is [0, 1], lower scores are better).

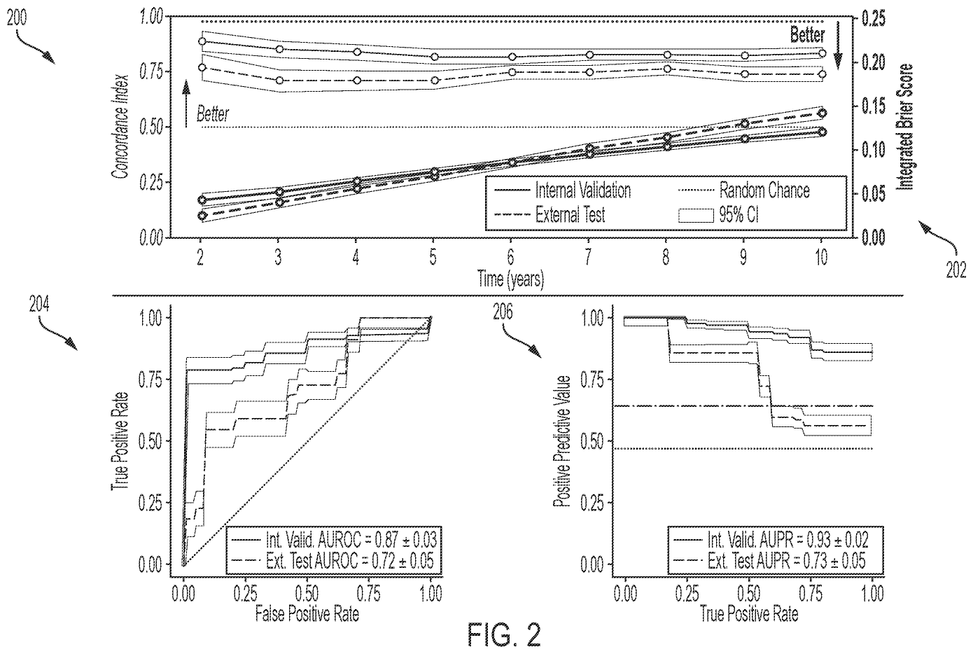

FIG. 2 depicts charts 202, 204, and 206 illustrating risk prediction performance of the reduction to practice. Chart 202 illustrates the concordance index (top), measuring reduction to practice risk discrimination—higher is better—and integrated Brier score (bottom), showing overall fit—lower is better—for various time points. Chart 204 depicts the receiver operator characteristic (ROC) curve at ten years for the internal validation and external test cohorts, with the respective areas under the curve (AUROC). Chart 206 illustrates the precision-recall (PR) curve at ten years for the internal validation and external test cohorts, with the respective areas under the curve (AUPR). For all charts 202, 204, 206, shaded areas represent approximate 95% confidence intervals, solid and dashed lines indicate the internal and external cohorts, respectively, and random chance performance thresholds are shown using dotted lines (in chart 206, a dot-dashed line is used to differentiate the internal random chance performance from the external). The chosen time of ten years was used to capture all sudden cardiac death from arrhythmia events in the population.

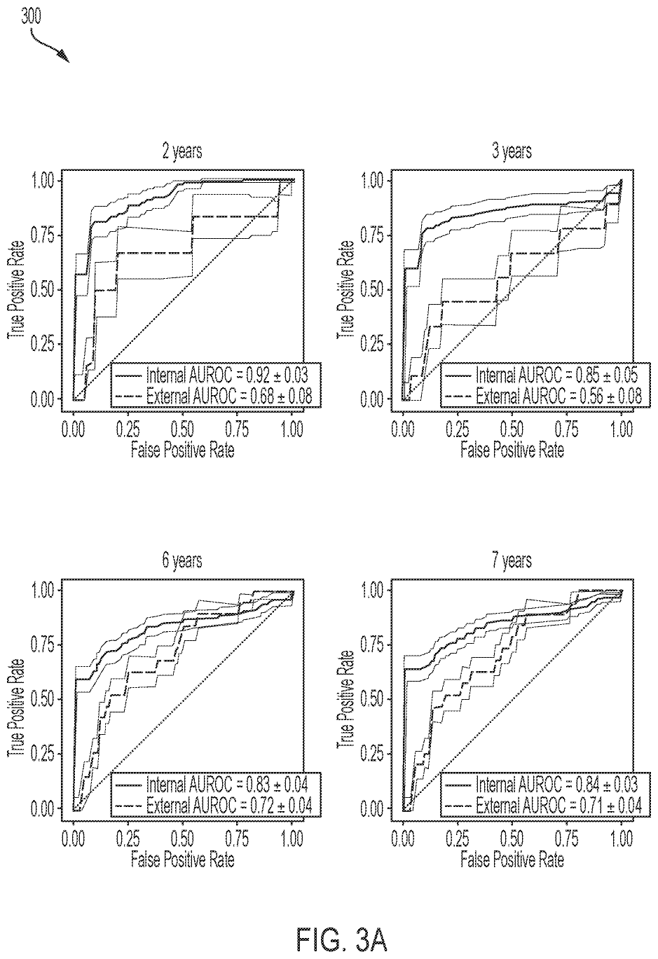

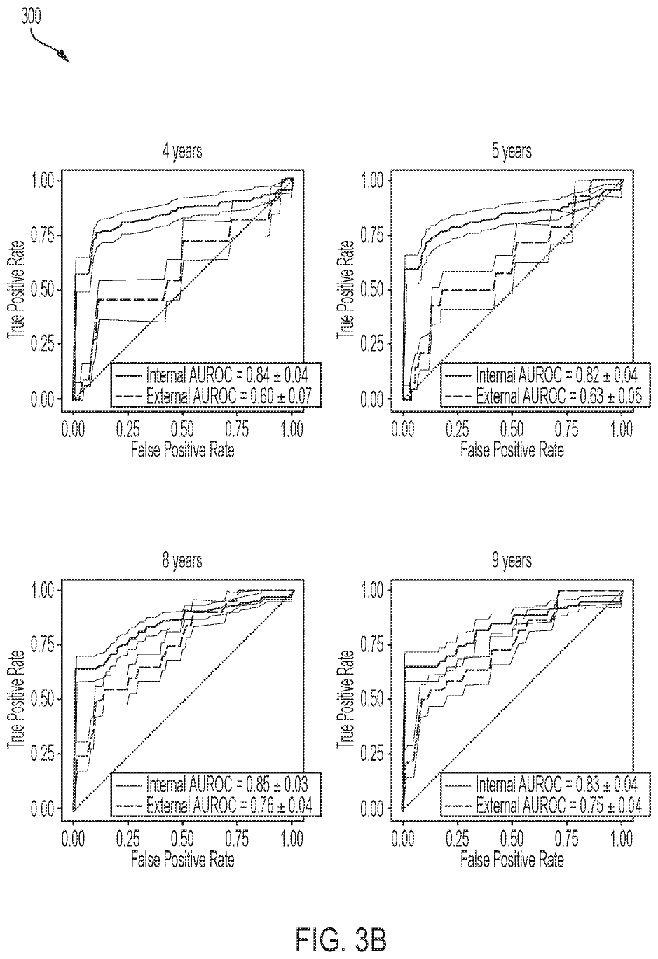

Chart 202 demonstrates that the reduction to practice has excellent concordance on the internal set (0.82-0.89) for events occurring before time T, where T ranges from 2 to 10 years. Additionally, the Brier score B̅s̅ ranges from 0.04 to 0.12, suggesting strong calibration, given the high concordance. The reduction to practice maintained its risk discrimination abilities at all times, as further evidenced by the results presented in reference to FIGS. 3A and 3B. In charts 204 and 206, all events up to ten years are used to construct the cross-validated receiver operator characteristic (ROC) and precision-recall (PR) curves for the internal validation set. For the internal validation set, the area under the ROC curve is 0.87 (95% Cl: 0.84-0.90), while the area under the PR curve is 0.93 (95% Cl: 0.91-0.95).

To demonstrate the reduction to practice's performance out-of-sample, an external test was performed using an independent, case-control set of 113 patients with coronary heart disease selected from participants with available CMR images enrolled in the PRE-DETERMINE study (see Section VII). These patients had less severe left ventricular systolic dysfunction, but otherwise had similar inclusion/exclusion criteria to those in the LVSPSCD study (see Section VII). Despite the dissimilarities between cohorts, the performance of the reduction to practice carried over well to the external cohort, resulting in a c-index of 0.71-0.77 and B̅s̅ of 0.03-0.14 as seen in chart 202 (dashed lines). Charts 204 and 206 show that the area under the ROC curve is 0.72 (95% Cl: 0.67-0.77) and the area under the PR curve is 0.73 (95% Cl: 0.68-0.78) on the external set.

FIGS. 3A and 3B depict charts 300 illustrating risk prediction performance of the reduction to practice. In particular, charts 300 depict results of the reduction to practice at various points in time, showing ROC curves for years 2-9 for the internal and external cohorts, with the respective areas under the curve (AUROC). Predicted outcomes are based on the estimated survival probability at the respective time points as computed from the survival probability function. Shaded areas represent approximate 95% confidence intervals, solid and dashed lines indicate the internal and external cohorts, respectively, and random chance performance thresholds are shown using dotted lines.

### III. Patient-Specific Survival Curves Predicted by the Reduction to Practice

The reduction to practice predicted cause-specific survival curves for each patient through two individualized parameters: the location μ and scale σ, characterizing the probability distribution of T_{SCDA}. In particular, μ represents the most likely T_{SCDA}, and σ provides a measure of confidence in the accuracy of μ. Using deep neural subnetworks to directly learn these parameters from CMR images and from clinical covariates in a way that best models the survival data produces highly-individualized survival probability predictions.

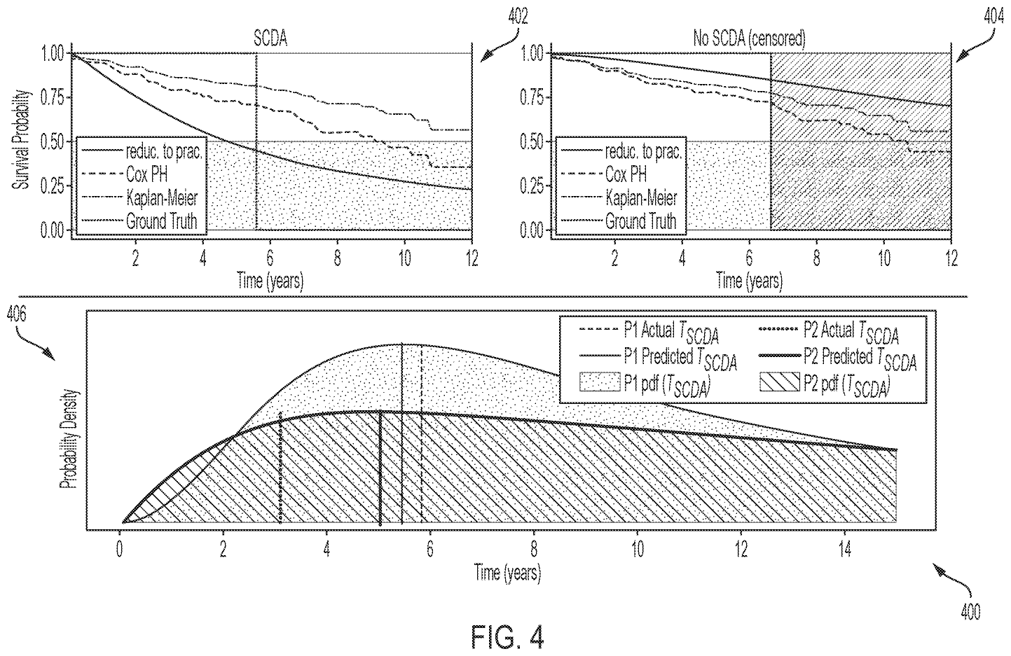

FIG. 4 depicts charts 402, 404, 406 illustrating individual patient survival predictions of the reduction to practice. Chart 402 shows a survival probability curve for an example patient in the external test set who experienced SCDA at around six years, and chart 404 shows a survival probability curve for an example patient in the external test set who did not experience SCDA but whose data was censored (e.g., not available) after about seven years, representing a non-SCDA event. Survival probability curves are plotted over time for the reduction to practice (solid curve), a representative Cox proportional hazards (Cox PH) model (dashed curve) on the clinical covariates, and a Kaplan-Meier estimator (dot-dashed curve), together with the indicator ground truth (dotted curve). As shown in both charts 402, 404, the survival curves estimated by the reduction to practice accurately predicted the event probabilities: in the first case, the estimated survival probability crosses the 50% threshold close to the event time; in the censored case, the reduction to practice predicts >80% probability of survival at the time of the (non-SCDA) event. For the patient with SCDA, the reduction to practice curve crosses the 50% survival probability threshold significantly closer to the SCDA time, as compared to the alternative curves, highlighting the reduction to practice's high calibration. For the censored patient (whose data included no SCDA), the reduction to practice estimates higher survival probability at the time of non-SCDA event compared to a different representative Cox-based technique. The representative technique thus demonstrates worse performance in comparison to the reduction to practice by underestimating the risk for the patient with SCDA and overestimating the risk for the censored patient.

Chart 406 depicts examples of the reduction to practice's predicted probability distributions for the time to SCDA (shaded areas, pdf (T_{SCDA})) for two patients in the external test set who experienced SCDA (P1 and P2). The predicted times to event (Predicted T_{SCDA}) are depicted as solid vertical lines (peaks of distributions); actual times (Actual T_{SCDA}) are depicted by dotted vertical lines.

In general, the predicted location parameter μ estimates the most probable T_{SCDA} and the predicted scale parameter σ provides a measure of confidence by characterizing the probability spread. The inclusion of both a location and a scale parameter in various embodiments offers the advantage of building uncertainty directly into the T_{SCDA} prediction. Importantly, this uncertainty is patient-specific and learned from data. In chart 406, P1 and P2 have different scale parameters, visualized as the widths of the distributions. Shown are the actual (dotted) and predicted (solid) T_{SCDA}, as well as the probability distributions (shaded). For P1, the prediction error is small (seen as the distance between the respective solid and dotted lines) and the reduction to practice is relatively certain, as seen by the narrower probability distribution of P1's T_{SCDA}, or, equivalently, a smaller predicted scale parameter. In the case of P2, the prediction error is larger (distance between the respective solid and dotted lines) and the reduction to practice predicts a wider distribution, or, equivalently, a larger scale parameter, indicating higher uncertainty. That is, the reduction to practice “recognizes” the inaccurate T_{SCDA} prediction and compensates for that by also predicting a more spread out distribution (larger scale parameter σ) for P2. Remarkably, using the entire internal cohort to quantify this direct relationship between prediction error—calculated as the relative mean absolute difference of actual and predicted times—and scale parameter reveals significant positive correlation (Pearson's r=0.42, p<0.001), demonstrating that the reduction to practice recognizes which predictions of T_{SCDA} will turn out inaccurate and “lowers the confidence” in them through a larger scale parameter. That is, the direct relationship between the prediction error and predicted scale parameter holds more generally for the entire dataset, indicating that the reduction to practice learns and is able to quantify the degree of inaccuracy in the T_{SCDA} prediction.

### IV. Cardiac Image Data Convolutional Subnetwork

This section details architecture, usage, and other aspects of a cardiac image data convolutional subnetwork (e.g., convolutional subnetwork 110 of FIG. 1) according to various embodiments.

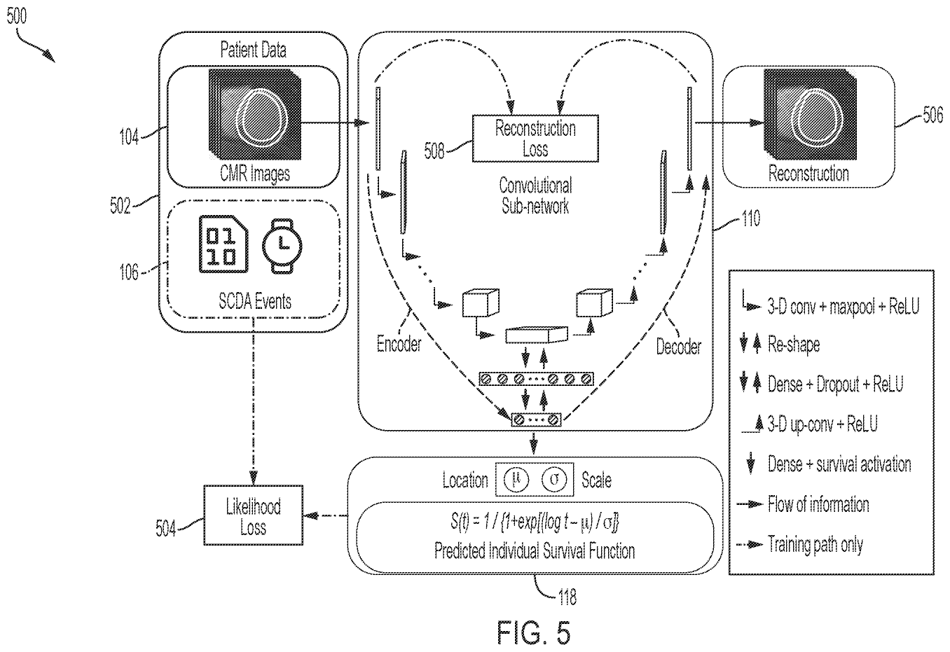

FIG. 5 is a schematic diagram of a convolutional subnetwork branch 500 according to various embodiments. As shown in FIG. 5, the convolutional subnetwork branch 500 includes convolutional subnetwork 110. Convolutional subnetwork 110 is trained using patient data 502 to provide survival probability data, e.g., location μ and scale σ, which are used to characterize patient-specific SCDA survival prediction data such as SCDA survival curve 118 (see Section VI). These elements are described in detail presently, by way of non-limiting example, in reference to the architecture used in the reduction to practice.

Patient data 502, which includes a portion of patient data 102, is used as training data by the convolutional subnetwork 110. Patient data 502 includes cardiac image data 104 and event data 106 as shown and described above in reference to FIG. 1. Cardiac image data 104 may include LGE CMR data, for which the left ventricle myocardium is automatically segmented. LGE CMR raw pixel values from the automatically segmented left ventricle are directly provided to the convolutional subnetwork 110, eliminating the need for arbitrary thresholds aiming to delineate areas of enhancement. LGE CMR is particularly suited for visualizing ventricle geometry and portions of the myocardium with contrast-enhanced remodelling from which convolutional subnetwork 110 learns image features most useful in predicting a patient's survival T_{SCDA}.

Labels for the cardiac image data 104 and associated with each patient are taken from the event data 106. Such labels include the observed time to the respective event and an indicator of whether the event was SCDA or otherwise (“otherwise” comprising, e.g., patient dropout or data unavailability). The labeled cardiac image data 104 forms a training corpus for training the convolutional subnetwork 110. During training (dot-dashed arrows), the neural network weights are optimized via a likelihood-based loss 504 and a reconstruction loss 508.

Convolutional subnetwork 110 may be implemented as, by way of non-limiting example, a 3-D convolutional neural subnetwork constructed using an encoder-decoder architecture, as was implemented in the reduction to practice. The encoder (left side of convolutional subnetwork 110) includes a sequence of 3-D convolutions, downsampling (maxpool), and rectified linear unit (ReLU) activation functions. The encoder may use a sequence of 3-D convolutions and pooling layers, followed by a dense layer to encode the original 3-D volume into a lower-dimensional vector. Nonlinear activation functions and dropout layers are included before each downsampling step. The encoder may be used for two purposes: survival and reconstruction.

For the survival path, the encoder may be first stratified into one of r (learned) risk categories (see Table 3) and then fed to a two-unit dense layer to predict—for each patient—survival probability data (e.g., location μ and scale σ), followed by an activation function. The activation function may clip In(μ) on the interval [−3, 3] and clip σ from below at θₘᵢₙ, where θₘᵢₙ may be determined such that the difference between the 95th and 5th percentiles of the predicted T_{SCDA} distribution is no less than a month. This survival activation function effectively restricts the “signal-to-noise” ratio μ/σ.

For the reconstruction path, the encoder may be decoded via a sequence of transposed convolutions to re-create the original volume. That is, the decoder (right side of convolutional subnetwork 110) may use transpose convolutions to reconstruct the original images, thereby ensuring that the encoded version of the CMR data is meaningful and able to reproduce the original images.

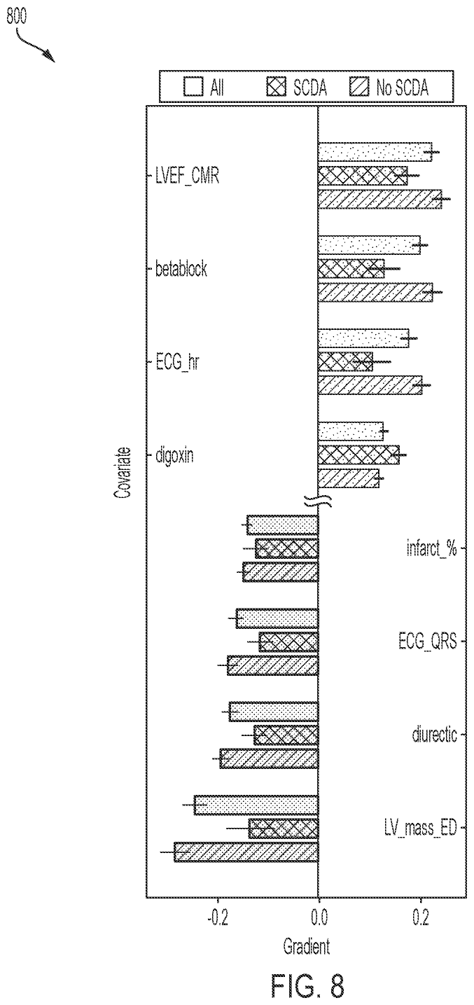

Detailed risk prediction performance for the convolutional subnetwork branch (CMR image data only) of the reduction to practice are described in reference to FIG. 8 and Table 1 presently.

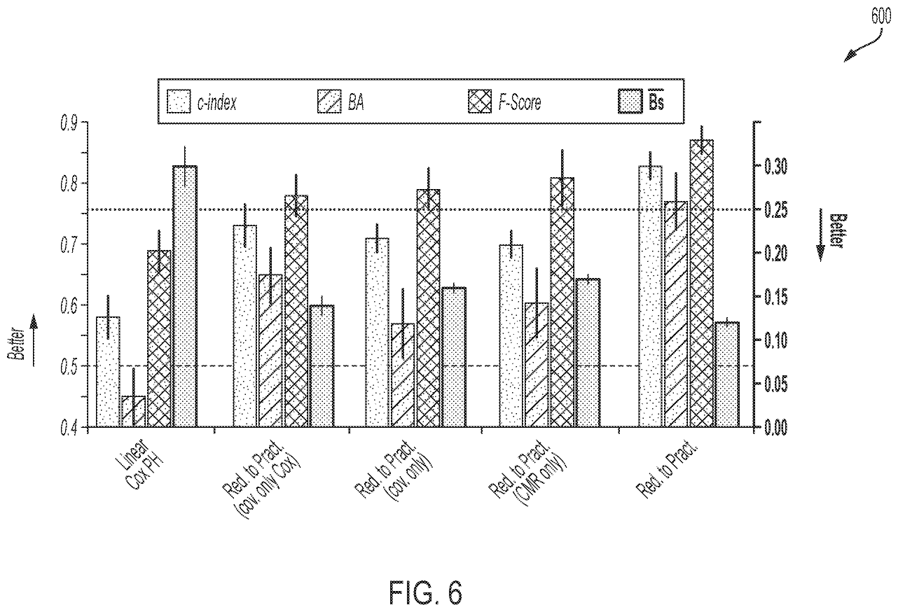

FIG. 6 depicts a graph 600 comparing the predictions of the convolutional subnetwork (and the dense subnetwork, see Section V) of the example reduction to practice, as well as the combined subnetworks of the entire reduction to practice, to a different representative technique. The four bars in each of the four groupings of four bars each represent, from left to right, a representative Cox proportional hazards model fit on the clinical covariates (Linear Cox PH), the dense subnetwork of the reduction to practice using clinical covariates with a Cox survival model (cov. only, Cox), the dense subnetwork of the reduction to practice using clinical covariates and the log-logistic survival model (cov. only), the convolutional subnetwork of the reduction to practice using cardiac magnetic resonance images only (CMR only), and the full prediction neural network of the reduction to practice. Random chance performance thresholds are shown using dotted lines. All performance measures are calculated using data up to T=10 years. All comparison values are based on averages over 100 cross-validation train/test splits of the internal validation data set. The error bars represent approximate 95% confidence intervals (see Section VI).

Each grouping of four bars depicts, from left to right, concordance index (c-index), balance accuracy (BA), F-score, and integrated Brier score (B̅s̅). The three leftmost bars in each grouping are relative to the left y-axis scale, and the rightmost bar in each grouping is relative to the right y-axis scale. Thus, graph 600 shows all-time performance using two measures: Harrell's c-index with the patient-specific μᵢ 's as the risk scores (exp(μᵢ) is the mode of the log-logistic distribution) to gauge risk discrimination ability, and integrated Brier score, which is defined as the time-average of mean squared error between true 0/1 outcome and predicted outcome probability and gauges both probability calibration and discrimination. Both measures are adjusted for censoring, corrected by weighing with the inverse probability of censoring, and calculated for data prior to a given cut-off time T; if unspecified, T=10 years, corresponding with the maximum event time in the data set. Metrics derived from the confusion matrix (e.g., precision and recall) are computed at several time points (T=2, 3 . . . years). Probability thresholds at these times were selected by maximizing F-score (for precision, recall, F-score) or Youden's J statistic (for sensitivity, specificity, balanced accuracy) on the training data.

Table 1 depicts detailed performance metrics for the reduction to practice relative to just the convolutional subnetwork of the reduction to practice. In particular, Table 1 depicts concordance index (c-index), integrated Brier score (B̅s̅), balanced accuracy (BA) and F-score for the entire reduction to practice (Red. to Pract.) and for the convolutional subnetwork using images only (CMR Only). All performance measures are calculated at T=10 years. The numbers for the internal cohort (Internal) are averages over 100 cross-validation splits and values on the external data set (External) represent a single evaluation on the entire set. In parentheses, approximate 95% confidence intervals are shown (see Section VI).

TABLE 1

| | Internal | | External | |
|---|---|---|---|---|
| | | Red. to Prac. | | Red. to Prac. |
| | Red. to Prac. | (CMR Only) | Red. to Pract. | (CMR Only) |
| c-index | 0.83 (0.81-0.85) | 0.70 (0.67-0.72) | 0.74 (0.71-0.77) | 0.63 (0.59-0.66) |
| B̅s̅ | 0.12 (0.12-0.13) | 0.17 (0.17-0.18) | 0.14 (0.14-0.15) | 0.19 (0.19-0.20) |
| BA | 0.77 (0.73-0.81) | 0.60 (0.55-0.65) | 0.73 (0.69-0.77) | 0.63 (0.58-0.68) |
| F-score | 0.87 (0.84-0.89) | 0.81 (0.77-0.85) | 0.65 (0.59-0.70) | 0.60 (0.55-0.65) |

As shown in Table 1, using only images as inputs, the reduction to practice achieved 0.70 (95% Cl: 0.67-0.72) c-index and 0.17 (95% Cl: 0.167-0.178) B̅s̅ for event data truncated at ten years on the internal validation set. On the external testing set, the image-only convolutional subnetwork achieved 0.63 (95% Cl: 0.59-0.66) c-index and 0.19 (95% Cl: 0.186-0.200) B̅s̅.

It is noteworthy that, although the dense subnetwork uses 22 clinical covariates and already includes engineered features from the CMR images (e.g., infarct size), the convolutional subnetwork using only CMR cardiac image data as inputs achieves similar performance to the dense one, as shown in FIG. 6. Furthermore, assembling the two subnetworks together using an ensembling subnetwork leads to a significant increase in overall performance compared to using just the covariate-based subnetwork, demonstrating that the convolutional subnetwork identifies completely different CMR-based features than the engineered ones.

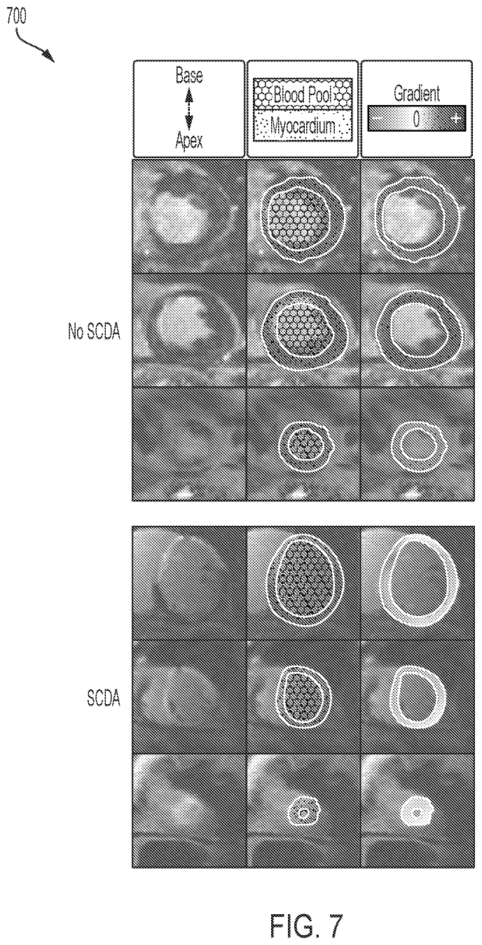

Imaging features learned by the convolutional subnetwork of the reduction to practice are interpreted using a gradient-based sensitivity analysis as illustrated in FIG. 7. The gradient quantifies the sensitivity of the predicted T_{SCDA} to the convolutional features learned by the subnetwork. Each resulting feature is scaled by the appropriate gradient to form the shown gradient maps. In particular, the trained network weights in the reduction to practice are interpreted for the convolutional subnetwork (and the dense subnetwork, see FIG. 8) using the gradients of outputs with respect to intermediary neural network internal representations of data. FIG. 7 adapts Grad-CAM to work on regression problems and applies it to the reduction to practice by performing a weighted average of the last convolutional layer feature maps, where the weights are averages of gradients of the location parameter output with respect to each channel. The result is then interpolated back to the original image dimensions and overlaid to obtain the gradient maps shown in FIG. 7, bottom row.

FIG. 7 depicts images 700 illustrating image convolutional subnetwork feature prediction according to the example reduction to practice. Images 700 show the convolutional subnetwork feature interpretation for an example patient who did not experience SCDA (No SCDA, top) and for a patient who did (SCDA, bottom). For each patient, contrast-enhanced short-axis cardiac magnetic resonance images (shown here are a subset of three locations in the heart, base to apex, top to bottom, left column) used as inputs by the reduction to practice are overlaid with blood pool and myocardium segmentation (middle column). A heat map of extracted features scaled by the value of the gradient shows contribution of the local pixel intensity to the predicted location parameter for the last convolutional layer (right column, heatmaps).

In detail, the features learned by the reduction to practice are depicted based on a gradient-based sensitivity analysis of the location parameter (μ, the most probable time to sudden cardiac death from arrhythmia, T_{SCDA}) to changes in the neural network input or features. The gradient value quantifies this sensitivity. The magnitude of the gradient measures the strength of the sensitivity of the predicted T_{SCDA} to inputs or intermediary features. The sign of the gradient shows the direction of the effect. That is, for a small increase in the value of inputs or features, a positive gradient indicates a higher predicted T_{SCDA}, whereas a negative gradient indicates a decrease in the predicted T_{SCDA}. Thus, myocardial regions found to be characterized with large positive gradient are interpreted as having high importance in increasing T_{SCDA} and, conversely, regions with large magnitude negative gradient represent areas that are responsible for decreasing the predicted T_{SCDA}.

The areas of contrast-enhanced myocardium (middle column) do not fully overlap with the gradient map, which suggests that while features learned by the convolutional subnetwork may co-localize with enhanced tissue, the convolutional subnetwork does not act as a mere enhancement locator. For example, though both patients have contrast-enhanced tissue, for the patient who did not experience SCDA, the effect of the contrast-enhanced tissue is to increase the predicted T_{SCDA}, suggesting a nuanced relationship between presence of enhancement and propensity of SCDA. That is, while the patient with SCDA shows high gradients in areas with contrast enhancement, indicating that such enhancements may indicate higher probability of SCDA, the patient who did not experience SCDA shows that the presence of enhancement can alternately lead to positive gradients, favorable for SCDA survival, suggesting that the subnetwork does not simply create a mask of the enhanced regions to make predictions, but learns a nuanced relationship between scar features and propensity for SCDA.

### V. Cardiac Covariate Data Dense Subnetwork

This section details architecture, usage, and other aspects of a cardiac covariate data dense subnetwork (e.g., dense subnetwork 114 of FIG. 1) according to various embodiments.

The dense subnetwork of the reduction to practice included a dense multilayer architecture. The dense subnetwork performed feature extraction from the clinical covariate data using a sequence of densely connected layers, followed by a dropout layer to prevent overfitting. The resulting tensor used a similar path to the one followed by the convolutional encoding of the convolutional subnetwork (see FIG. 5) to eventually map survival probability data in the form of the location (u) and scale (o) parameters. The dense subnetwork thus discovered and extracted potential nonlinear relationships between the covariates and integrated them within the reduction to practice's overall survival predictions.

Labels for the covariate data 108 and associated with each patient are taken from the event data 106. Such labels include the observed time to the respective event and an indicator of whether the event was SCDA or otherwise (“otherwise” comprising, e.g., patient dropout or data unavailability). The labeled covariate data 108 forms a training corpus for training the covariate subnetwork 114. During training (dot-dashed arrows), the neural network weights are optimized via a likelihood-based loss 504 and a reconstruction loss 508.

Graph 600 of FIG. 6 compares the performance of the dense subnetwork of the reduction to practice to a representative (linear) Cox proportional hazards model. As with the convolutional subnetwork, the trained network weights in the reduction to practice are interpreted using the gradients of outputs with respect to intermediary neural network internal representations of data. For the dense subnetwork, the gradient of the location parameter output is taken with respect to each of the inputs and averaged over three groups: all patients, patients with SCDA, patients with no SCDA. To avoid mis-attributing performance differences to the underlying statistical models, the neural network loss function is replaced with that used in a Cox proportional hazards model. Using clinical covariate data only, the reduction to practice with a Cox survival model (cov. only, Cox) outperforms the standard Cox proportional hazards model (Linear Cox PH) in terms of c-index (0.73 vs. 0.58, no hash, left y-axis), balanced accuracy (0.65 vs. 0.45, diagonal hash, left y-axis), F-score (0.78 vs 0.69, cross hash, left y-axis), and B̅s̅ (0.14 vs 0.30, red, no hash, right y-axis).

The covariate data used in the dense subnetwork may represent independent variables that can influence the SCDA prediction, but that are not of direct interest. The 22 cardiac covariates that were used in the covariate dense subnetwork of the reduction to practice for both the internal validation data and the external independent test set data are presented in Table 2, grouped according to category (demographics, risk factors, CMR measurements, ECG measurements, and medication use). In general, embodiments may incorporate any, or a combination, of variables from any, or a combination, of these or other categories.

TABLE 2

| | Internal | | External | | |
|---|---|---|---|---|---|
| | (n = 156) | | (n = 113) | | P-Value |
| Demographics | | | | | |
| Age, y | 61 | (+11) | 62 | (+11) | 0.443 |
| Male sex | 135 | (87) | 98 | (87) | 0.483 |
| White | 126 | (81) | 95 | (84) | 0.345 |
| Risk Factors | | | | | |
| Tobacco use | 104 | (67) | 83 | (73) | 0.117 |
| DM | 51 | (33) | 44 | (39) | 0.146 |
| Hypertension | 105 | (67) | 79 | (70) | 0.326 |
| Hyperlipidemia | 121 | (78) | 106 | (94) | p < .001 |
| EF non-CMR, % | 25 | (+7) | 39 | (+13) | p < .001 |
| Duration of CM, y | 5 | (+6) | 5 | (+7) | 0.920 |
| CMR Measurements | | | | | |
| LVEF, % | 28 | (+8) | 36 | (+11) | p < .001 |
| LV mass (ED), g | 146 | (+45) | 127 | (+35) | p < .001 |
| Infarct size, % | 28 | (+14) | 16 | (+10) | p < .001 |
| ECG Measurements | | | | | |
| Heart rate | 70 | (+12) | 69 | (+14) | 0.748 |
| Presence of LBBB | 24 | (15) | 5 | (4) | 0.002 |
| History of atrial fib. | 29 | (19) | 4 | (4) | p < .001 |
| QRS duration, ms | 116 | (+27) | 104 | (+24) | p < .001 |
| Medication Use | | | | | |
| β-blocker | 146 | (94) | 103 | (91) | 0.227 |
| ACE inhibitor or ARB | 141 | (90) | 94 | (83) | 0.040 |
| Lipid-lowering | 142 | (91) | 105 | (93) | 0.289 |
| Diuretic | 80 | (51) | 58 | (51) | 0.497 |
| Antiarrhythmic drug | 13 | (8) | 2 | (2) | 0.010 |
| Digoxin | 20 | (13) | 2 | (2) | p < .001 |
| Outcome | | | | | |
| SCDA event | 41 | (26) | 22 | (19) | 0.097 |
| Time to Event, y | 6 | (+3) | 7 | (+3) | 0.004 |

In Table 2, for continuous variables, the values are depicted as mean (±std. dev.), and for categorical variables, the values are depicted as count (% of total). P-values are based on two-sample Welch's t-test for continuous variables and Mann-Whitney U test for categorical variables. The abbreviations appearing in Table 2 include: SCDA, sudden cardiac death from arrhythmia; DM, diabetes mellitus; EF, ejection fraction; CMR, cardiac magnetic resonance; CM, cardiomyopathy; LVEF, left ventricular ejection fraction; LV, left ventricle; ED, end-diastolic; LBBB, left bundle branch block; and fib.; fibrillation. In the reduction to practice, the CMR measurements were obtained for CMR imaging separate from the cardiac image data; however, in various embodiments, they may be obtained from the cardiac image data or elsewhere.

FIG. 8 depicts a chart 800 illustrating dense subnetwork covariate interpretation according to the example reduction to practice. In particular, chart 800 represents a sensitivity analysis of the predicted T_{SCDA} with respect to changes in the covariates, demonstrating interpretability of the dense subnetwork. Chart 800 shows data for an average of all patients (solid bars), patients with SCDA (square hash bars), and patients with no SCDA (diagonal hash bars), with covariates for the top four highest (top right) and bottom four lowest (bottom left) average gradients of the subnetwork output (i.e., the predicted location parameter) with respect to the clinical covariate inputs shown. The abbreviations used in FIG. 8 are: LVEF_CMR, left ventricular ejection fraction computed from CMR; betablock, use of β-blocker medication; ECG_hr, heart rate from ECG; digoxin, use of Digoxin medication; infarct %, infarct size as a % of total volume; ECG_QRS, QRS complex duration from ECG; and LV_mass_ED, left ventricular mass in end diastole. High positive gradients (top right) denote covariates for which small increases in value lead to large increases in T_{SCDA}, whereas small negative gradients (bottom left) represent covariates for which small increases in value lead to large decreases in T_{SCDA}. The top four positive gradient covariates are left ventricular ejection fraction computed from CMR, β-blocker medication, heart rate computed from ECG, and use of Digoxin. The bottom four negative gradient covariates are left ventricular mass at end-diastole, use of diuretic medication, QRS duration computed from ECG, and infarct size (%).

### VI. Training and Validation

Training an overall embodiment may proceed, by way of non-limiting example, according to the training of the reduction to practice, described presently. Once the cardiac image convolutional subnetwork and the cardiac covariate dense subnetwork of the reduction to practice were trained, they were frozen and joined using a learned linear combination ensembling subnetwork to combine the survival probability data, which for the reduction to practice were location μ and scale σ.

The survival probability data aimed to minimize a loss function, which for the reduction to practice was in the form of a negative log likelihood function for the log-logistic distribution, described presently, accounting for censoring in the data and class imbalance. For each patient i, the outcome data was the pair (Xᵢ, Δᵢ), where Xᵢ represents the minimum between the time to SCDA from arrhythmia Tᵢ and the (right) censoring time Cᵢ after which either follow-up was lost or the patient died due to a non-SCDA risk (each patient was associated with only one of Tᵢ or Cᵢ). The outcome Δᵢ was 1 if the patient had the arrhythmic event before they were censored (Tᵢ≤Cᵢ) and 0 otherwise. The (pseudo-) survival probability function Sᵢ/(t), the probability that the time to SCDA exceeds t, was estimated. The Tᵢ's were modeled as independent, each having a cause-specific hazard rate based on the log-logistic distribution with location parameter uj and scale parameter σᵢ, such that Sᵢ(t; μᵢ, σᵢ)=1/{1+exp[(log t−uᵢ)/σᵢ]}. The patient-specific parameters μᵢ and σᵢ were modeled as outputs of the two subnetworks, trained by minimizing the loss function given by the negative likelihood:

$$- log ℒ = - {\sum }_{i} \frac{log {x}_{i} - {μ}_{i}}{{σ}_{i}} + {δ}_{i} log {σ}_{i} + ( 1 + {δ}_{i} ) log [ 1 + exp ( \frac{log {x}_{i} - {μ}_{i}}{{σ}_{i}} ) ]$$

In the above loss function, xᵢ represents the observed time and δᵢ represents the censoring status. With μᵢ and σᵢ estimated, the patient-specific survival functions were given by Sᵢ/(t) as above.

The reconstructed output of the convolutional subnetwork minimized the mean squared error (MSE) to the original input. Its contribution to total loss was learned to provide regularization to the imaging features extracted, ensuring the survival fit relied on features able to reconstruct the original image. Both stochastic gradient descent (SGD) and Adam optimizers (Kingma, D. P. & Ba, J. Adam: A method for stochastic optimization, arXiv preprint arXiv: 1412.6980 (2014)) were used for the reduction to practice. All code was developed in Python using Keras and Tensorflow. Each train/evaluate fold took 3-5 minutes on an NVIDIA Titan RTX graphics processing unit.

The reduction to practice development and internal validation were performed using the LVSPSCD cohort (see Section VII). Following a hyperparameter tuning step, the best architecture was then used on the entire internal validation set to find the best neural network weights. As the ensembling subnetwork was hyperparameter-free, it did not use hyperparameter tuning.

For hyperparameter tuning of the reduction to practice, a hyperparameter search was performed using the set of parameter values described in Table 3, given the vast number of hyperparameter configurations available to define the architectures. The package hyperopt was used to sample parameter configurations from the search space using the Parzen window algorithm to minimize the average validation loss resulting from a stratified ten-times repeated ten-fold cross-validation process. The maximum number of iterations was 300 for the dense subnetwork and lowered to 100 for the convolutional subnetwork, given its highly increased capacity. Each fold was run using early stopping based on the loss value on a withheld 10% portion of the training fold with a maximum of 2000 epochs (20 gradient updates per epoch). In hyperparameter tuning, systems were optimized using SGD with a learning rate of 0.01. The architecture with the highest Harrell's concordance index was selected.

TABLE 3

| | Convolutional | Dense |
|---|---|---|
| Parameter | Subnetwork [Choice] | Subnetwork [Choice] |
| Epochs (maximum) | 2000 | 2000 |
| Learning rate (initial) | .01 | .01 |
| Batch size | 32 | 32 |
| Steps per epoch | 20 | 20 |
| Activation function | ReLU | ReLU |
| Convolutional | 3 × 3 × 3 | N/A |
| kernel size | | |
| Network depth | {2, 4} [2] | {1, 2, 3, 4, 5} [3] |
| Risk categories | {1, 2, 3} [2] | {1, 2, 3} [1] |
| Dropout | Uniform(0, 0.5) [0.04] | Uniform(0, 0.5) [.22] |
| I1 (bias and kernel) | LogUniform(10⁻⁷, | LogUniform(10⁻⁷, |
| regularization | 10⁻³) [4.02 × 10⁻⁵] | 10⁻³) [1.41 × 10⁻⁵] |
| I2 (bias and kernel) | LogUniform(10⁻⁶, | LogUniform(10⁻⁶, |
| regularization | 10⁻²) [3.25 × 10⁻⁶] | 10⁻²) [8.27 × 10⁻⁵] |
| Convolutions per | {1, 2} [1] | N/A |
| sampling level | | |
| Convolutional | {8, 12, 16, 20, 24} [24] | N/A |
| filters (initial) | | |
| Reconstruction | Uniform(0, 10) [.06] | N/A |
| loss weight | | |
| Latent representation | Uniform{10, 30} [22] | N/A |
| dimension | | |
| Units per dense layer | N/A | Uniform{4, 24} [8] |

As shown in Table 3, the hyperparameters used to define network architectures and training guidelines (left column) were optimized by randomly sampling using the Parzen windows algorithm from the search spaces defined for the convolutional (middle column) and dense subnetworks (right column). In Table 3, square braces denote equally probable sampling from the set. “Uniform” refers to uniformly distributed either continuously (parentheses) or discretely (braces), and “LogUniform” refers to uniformly distributed exponent. No qualification means the hyperparameter was fixed. “Network depth” is the number of convolution/downsample steps for the convolutional subnetwork and the number of dense layers for the dense subnetwork. “Risk categories” refers to the number of strata used to divide before the final ensembling subnetwork. The “Latent representation dimension” is the number of units of the dense layer immediately following the last convolution/downsample step. The hyperparameter was stopped after 100 and 300 iterations for the convolutional and dense subnetworks, respectively. Choices for the final reduction to practice are displayed in bold in square brackets.

Once fully trained, the internal performance of the reduction to practice was assessed using ten repetitions of stratified ten-fold cross-validation on the LVSPSCD cohort (see Section VII). Early stopping based on the c-index on a withheld 10% subset was implemented with a maximum training of 2000 epochs (20 gradient updates). The optimizer was Adam with learning rate 10-5 for the convolutional subnetwork, 5×10−4 for the dense subnetwork, and 0.01 for the ensembling subnetwork. A final reduction to practice was trained with all the available LVSPSCD data and tested on the PRE-DETERMINE cohort (see Section VII). To estimate confidence intervals on the external cohort, the same cross-validation process was applied to the PRE-DETERMINE cohort, supplementing the training data in each fold with the LVSPSCD cohort. Approximate normal confidence intervals were constructed using the 100 folds.

All values reported herein on the internal validation data set were averages over 100 data splits resulting from a ten-times repeated ten-fold stratified cross-validation scheme. Values reported on the external test data set represented a single evaluation on the entire set. All confidence intervals were normal approximations resulting from the aforementioned 100 splits. In computing confidence intervals for the external test set, the same procedure was used on all available data, ensuring test folds came exclusively from the external data set. Error bars are standard errors with sample standard deviation estimated from the 100 splits. Correlation P-value was based on the exact distribution under the bivariate normal assumption. Covariate P-values are based on two-sample Welch's t-test for continuous variables and Mann-Whitney U test for categorical variables. Cox proportional hazards analysis was performed using the Python lifelines package, it included a hyperparameter sweep for the l₁ and l₂ regularization terms, and followed the same train/test procedure.

### VII. Reduction to Practice Data Sources, Data Acquisition and Data Preparation

The reduction to practice included a retrospective analysis based on a subset (n=269) of patients selected from the prospective clinical trials described presently. Of note is that the entire reduction to practice development was based on the internal cohort, while the external cohort was used exclusively for testing (outcomes were solely used for computing relevant metrics once the reduction to practice was fixed).

Patient data for the internal cohort came from the Left Ventricular Structural Predictors of Sudden Cardiac Death Study (LVSPSCD) (ClinicalTrials.gov ID NCT01076660) sponsored by Johns Hopkins University. As previously described in Wu, K. C. et al., Baseline and dynamic risk predictors of appropriate implantable cardioverter defibrillator therapy, J. Am. Hear. Assoc. 9, e017002 (2020) and Schmidt, A. et al., Infarct tissue heterogeneity by magnetic resonance imaging identifies enhanced cardiac arrhythmia susceptibility in patients with left ventricular dysfunction, Circulation 115, 2006-2014, DOI: 10.1161/CIRCULATIONAHA, 106.653568 (2007), patients satisfying clinical criteria for ICD therapy for SCDA (left ventricle ejection fraction≤35%) were enrolled at 3 sites: Johns Hopkins Medical Institutions (Baltimore, MD), Christiana Care Health System (Newark, DE), and the University of Maryland (Baltimore, MD). A total of 382 patients were enrolled between November 2003 and April 2015. Patients were excluded if they had contraindications to CMR, New York Heart Association (NYHA) functional class IV, acute myocarditis, acute sarcoidosis, infiltrative disorders (e.g., amyloidosis), congenital heart disease, hypertrophic cardiomyopathy, or renal insufficiency (creatinine clearance <30 mL/minute after July 2006 or <60 mL/minute after February 2007). The protocol was approved by the institutional review boards at each site, and all participants provided informed consent. CMR imaging was performed within a median time of 3 days before implantable cardioverter device (ICD) implantation. The current study focused on the ischemic cardiomyopathy patient subset with adequate late gadolinium enhanced (LGE)-CMR, totaling 156 patients. As part of the clinical study, the participants had undergone single-chamber or dual-chamber ICD, or cardiac resynchronization with an ICD (CRT-D) implantation based on current guidelines. The programming of antitachycardia therapies was left to the discretion of the operators.

Patient data for the external cohort came from the PRE-DETERMINE (ClinicalTrials.gov ID NCT01114269) and accompanying DETERMINE Registry (ClinicalTrials.gov ID NCT00487279) study populations, which were multicenter prospective cohort studies comprised of patients with coronary disease on angiography or documented history of myocardial infarction (MI). The PRE-DETERMINE study enrolled 5764 patients with documented MI and/or mild to moderate LV dysfunction based on left ventricle ejection fraction (LVEF), (LVEF between 35-50%), who did not fulfill consensus guideline criteria for ICD implantation on the basis of LVEF and NYHA class (i.e., LVEF >35% or LVEF between 30%-35% with NYHA Class I HF) at study entry. Exclusion criteria included a history of cardiac arrest not associated with acute myocardial infarction, current or planned ICD, or life expectancy <6 months. The accompanying DETERMINE Registry included 192 participants screened for enrollment in PREDETERMINE who did not fulfill entry criteria on the basis of having an LVEF <30% (n=99), LVEF between 30%-35% with NYHA Class II-IV heart failure (n=19), or an ICD (n=31) or were unwilling to participate in the biomarker component of PREDETERMINE (n=43). Within these cohorts, 809 participants had LGE CMR imaging performed. Within this subset of patients, 23 cases of SCD occurred and were matched to four controls on age, sex, race, LVEF and follow-up time using risk set sampling. Out of the resulting 115 patients, the reduction to practice focused on 113 patients with adequate LGE CMR images for analysis. Finally, covariate data for this cohort were minimally “harmonized” with the internal cohort, by retaining common covariates only. Some significant differences between the external and internal cohorts remained, such as significantly higher LVEF in the external cohort.

The LGE CMR images in the internal and external cohort were acquired using 1.5-T magnetic resonance imaging devices (Signa, GE Medical Systems, Waukesha, Wisconsin; Avanto, Siemens, Erlangen, Germany). All were 2-D parallel short-axis left ventricle stacks. The LGE contrast agent used was 0.15-0.20 mmol/kg gadodiamide (Omniscan, GE Healthcare) or gadopentetate dimeglumine (Magnevist, Schering AG) and the scan was captured 10-30 minutes after injection. Due to the multi-center nature of the clinical studies considered here, there were variations in CMR acquisition protocols. The most commonly used sequence was inversion recovery fast gradient echo pulse, with an inversion recovery time typically starting at 250 ms and adjusted iteratively to achieve maximum nulling of normal myocardium. Typical spatial resolutions ranged 1.5−2.4×1.5−2.4×6−8 mm, with 2-4 mm gaps. LGE CMR images in the external cohort was sourced from sixty sites with a variety of imaging protocols, whereas those in internal cohort originated from three sites and were more homogeneous.

In both the LVSPSCD and PRE-DETERMINE/DETERMINE cohorts, baseline data on demographics, clinical characteristics, medical history, medications, lifestyle habits, and cardiac test results were collected (see Table 2 for a list of the common ones between the cohorts that were used in the reduction to practice). The primary endpoint for LVSPSCD was SCDA defined as therapy from the ICD for rapid ventricular fibrillation or tachycardia, or a ventricular arrhythmia not corrected by the ICD. For the PRE-DETERMINE studies, the primary end point was sudden and/or arrhythmic death. Deaths were classified according to both timing (sudden versus non-sudden) and mechanism (arrhythmic versus non-arrhythmic). Unexpected deaths due to cardiac or unknown causes that occurred within one hour of symptom onset or within 24 hours of being last witnessed to be symptom free were considered sudden cardiac deaths. Deaths preceded by an abrupt spontaneous collapse of circulation without antecedent circulatory or neurological impairment were considered arrhythmic in accordance with the criteria outlined by Hinkle and Thaler. Deaths that were classified as non-arrhythmic were excluded from the endpoint regardless of timing. Out-of-hospital cardiac arrests due to ventricular fibrillation that were successfully resuscitated with external electrical defibrillation were considered aborted arrhythmic deaths and included in the primary endpoint.

The training data for the reduction to practice included the unprocessed LGE CMR scans and the clinical covariates listed in Table 2. The training targets were the event time and event type (SCDA or non-SCDA). As a pre-processing step, the raw LGE CMR scans were first segmented for left ventricle myocardium using a method based on convolutional neural networks as described in Abramson, H. G. et al., Anatomically-informed deep learning on contrast-enhanced cardiac MRI for scar segmentation and clinical feature extraction, arXiv preprint, arXiv: 2010.11081 (2020). Briefly, this segmentation network included three subnetworks: a U-net with residual connections trained to identify the entire region of interest, a U-net with residual connections trained to delineate the myocardium wall, and an autoencoder tasked with correcting anatomical inaccuracies that may have resulted in the segmentation. In this context, anatomical correctness was defined via a list of pass/fail rules (e.g., no holes in the myocardium, circularity threshold, no disconnected components, etc.). Once each patient's LGE CMR images were segmented via this method, all voxels outside the left ventricle myocardium were zeroed out and the slices were sorted apex-to-base using DICOM header information and step-interpolated on a regular 64×64×12 grid with voxel dimensions 2.5×2.5×10 mm. These dimensions were chosen to make all patient volumes consistent with minimal interpolation from the original resolution, while allowing enough room to avoid truncating the left ventricle. Finally, the cardiac image data input to the reduction to practice included a two-channel image. The first channel was a one-hot encoding of the myocardium and blood pool masks. The second channel had zeros outside of the myocardium and the original CMR intensities on the myocardium, linearly scaled by multiplication with half the inverse of the median blood pool intensity in each slice. To mitigate overfitting, train-time data augmentation was performed on the images, specifically 3-D in-plane rotations in increments of 90° to avoid artifacts, and panning of the ventricle within the 3-D grid. The clinical covariate data was de-meaned and scaled by the standard deviation.

### VIII. Conclusion: Features, Advantages, and Variations

Embodiments provide an entirely novel approach to SCDA risk assessment based on specialized neural subnetworks to predict patient-specific SCDA survival prediction data. The subnetworks can include a 3-D convolutional subnetwork operating on raw unsegmented LGE CMR images that visualize heart disease-induced scar distribution, a dense fully-connected subnetwork operating on clinical covariates, and an ensembling subnetwork that combines survival probability data produced by the other subnetworks. The predicted patient-specific survival curves of a reduction to practice offered accurate SCDA probabilities at all times up to ten years.

Some embodiments are not only highly flexible, able to capture complex imaging and non-imaging feature inter-dependencies, but are also robust owing to the statistical framework governing the way these features are combined to fit the survival data. Some embodiments predict entire probability distributions for the T_{SCDA}, allowing for uncertainties in predictions to be themselves patient-specific and learned from data, thereby equipping such embodiments with a self-correction mechanism. This approach remedies a well-known significant limitation of neural networks, the high confidence in erroneous predictions.

Some embodiments provide solutions to problems of non-deep-learning-based prior art techniques, e.g., the underutilization of image data, the need time-consuming, manual processing steps, typically involving arbitrarily chosen image intensity thresholds, and the usage of features that either too coarse to capture the intricacies of the scar distribution, or highly mathematical, undermining their physiological underpinning. Further, some embodiments provide solutions to problems of prior art deep learning techniques, e.g., focus on cardiologist-level detection in ECG signals.

Some embodiments operate on both raw, unsegmented LGE CMR images and clinical covariates within a unified feature learning process, allowing for the different data types to synergistically inform overall survival. Some embodiments utilize such data to automatically identify features that best model and predict the T_{SCDA}. The clinical covariates according to some embodiments include standard, manually derived imaging features, which prevents the convolutional subnetwork from merely re-discovering these known features, and instead encourages it to learn new features. Some embodiments achieve performance levels that are beyond the state-of-the-art in both relative terms—SCDA risk ordering among patients—as well as absolute—accurately calibrated probabilities of SCDA.

The robust testing scheme applied to the reduction to practice demonstrates that it overcame significant limitations of previous work on SCDA risk. For example, the reduction to practice demonstrated high generalizibility through the use of internal cross-validation performance numbers resulting from 100 train/test splits of the data and on an entirely separate external cohort, with only modest performance degradation on the latter. Further, the reduction to practice's computation of performance metrics at multiple time points up to ten years prevented it from being over-tuned to a certain time horizon.

In comparison to a representative regularized Cox proportional-hazards model using the available clinical covariates to serve as a baseline for the rest of the analysis, the reduction to practice, which utilized the same covariates, performed significantly better in the same proportional-hazards setting, highlighting the advantages of nonlinear relationships in the covariates. Furthermore, even when restricted to only LGE CMR images to predict arrhythmia risk, the convolutional subnetwork in the reduction to practice: (1) outperformed the Cox proportional-hazards model constructed using clinical covariates that included standard imaging and non-imaging features, and (2) performed on par with the covariate-only subnetwork of the reduction to practice using the same clinical variables, suggesting that the image-only subnetwork in the reduction to practice was able to identify highly predictive imaging features in the LGE CMR images. Finally, the complete, ensembled reduction to practice's superior performance to either of its convolutional or dense subnetworks alone demonstrates that the novel imaging features found by the reduction to practice's convolutional subnetwork cannot be explained away even when considering nonlinear relationships between standard covariates.

Interpretability of artificial intelligence algorithms is paramount to their broad adoption, and concerns surrounding it are particularly prevalent in healthcare. Some embodiments provide a level of interpretability that is embedded in the overall design and arrangement of the subnetworks. The sensitivity analysis of the outputs to the extracted features disclosed herein offers a lens into the neural network, rendering some transparency to the algorithm “black-box”. In addition, CMR images taken as input by the convolutional subnetwork of the reduction to practice were automatically segmented to include myocardium-only raw intensity values, and the subnetwork was designed as an encoder-decoder, by way of non-limiting example, to ensure minimal loss of information during the feature extraction process.

The reduction to practice achieved strong performance despite working on a relatively small data set. A concern with deep learning on smaller data sets is overfitting, which manifests itself as high performance during training (good fit), but poor performance when applied to a new test set. Indeed, the results disclosed herein show some differences between metrics on the internal validation and external test cohorts. However, although the two cohorts' covariates were “harmonized” where possible, they represented two different distributions (e.g., low versus moderately reduced left ventricle ejection fraction, unmatched versus matched case-control, three versus 60 CMR acquisition sites, etc.). Furthermore, several measures were taken to mitigate overfitting in the reduction to practice: in addition to standard techniques—dropout, kernel and bias regularizers—the convolutional subnetwork was implemented as an autoencoder that uses the distilled features used in risk prediction to also re-construct the original image as an additional regularization technique. Finally, the numbers cited on the internal validation set are averages of the test performance of hundreds of train/test data splits, adding a layer of statistical rigor.

Further embodiments may operate on additional all-cause mortality data, accounting for other types of death that may occur. Such embodiments may directly model competing risks and compute a cause-specific cumulative incidence function.

Further embodiments may operate on any of a variety of covariates for the covariate data utilized by the dense subnetwork. For example, such embodiments may incorporate additional covariates that have been identified as predictors of SCDA, but that are not part of standard clinical practice.

Certain embodiments can be performed using a computer program or set of programs executed by an electronic processor. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

1. A computer-implemented neural network method of predicting patient-specific arrhythmic sudden cardiac death survival, the method comprising:
obtaining cardiac image data for a patient;
obtaining cardiac covariate data for the patient;
providing the cardiac image data to a first subnetwork;
providing the cardiac covariate data to a second subnetwork;
combining an output from the first subnetwork with an output from the second subnetwork, wherein survival probability data is provided; and
outputting arrhythmic sudden cardiac death survival prediction data based on the survival probability data, wherein the arrhythmic sudden cardiac death survival prediction data is specific to the patient.

2. The method of claim 1, further comprising treating the patient with an implantable cardioverter device based on the sudden cardiac death survival prediction data.

3. The method of claim 1, wherein the arrhythmic sudden cardiac death survival prediction data comprises an arrhythmic sudden cardiac death survival curve for the patient.

4. The method of claim 1, wherein the arrhythmic sudden cardiac death survival prediction data comprises a probability distribution representing time of predicted arrhythmic sudden cardiac death.

5. The method of claim 1, wherein the survival probability data comprises a probability distribution location datum and a probability distribution scale datum.

6. The method of claim 5, further comprising outputting a patient-specific uncertainty assessment for the arrhythmic sudden cardiac death survival prediction data based on the probability distribution scale datum.

7. The method of claim 1, wherein the combining comprises providing the first output and the second output to a third subnetwork that provides the survival probability data.

8. The method of claim 1, wherein the cardiac covariate data for the patient comprises cardiac image measurement data, risk factor data, electrocardiogram data, and medication data for the patient.

9. The method of claim 8,
wherein the cardiac image measurement data comprises at least one of left ventricle mass data or infarct size data,
wherein the risk factor data comprises ejection fraction data,
wherein the electrocardiogram data comprises at least one of heart rate data or QRS complex data, and
wherein the medication data comprises at least one of beta blocker medication data, diuretic medication data, digoxin medication data.

10. The method of claim 1, wherein the first subnetwork comprises an encoder-decoder subnetwork, and wherein the second subnetwork comprises a dense subnetwork.

11. The method of claim 1,
wherein the first subnetwork is trained with training cardiac image data, the training cardiac image data comprising times until arrhythmic sudden cardiac death events and times until non-arrhythmic-sudden-cardiac-death events, and
wherein the second subnetwork is trained with training cardiac covariate data, the training cardiac covariate data comprising times until arrhythmic sudden cardiac death events and times until non-arrhythmic-sudden-cardiac-death events.

12. A computer-implemented neural network system for predicting patient-specific arrhythmic sudden cardiac death survival, the system comprising:
a first subnetwork that accepts cardiac image data for a patient and provides corresponding first survival probability data;
a second subnetwork that accepts cardiac covariate data for a patient and provides corresponding second survival probability data; and
an output that provides arrhythmic sudden cardiac death survival prediction data based on the first survival probability data and the second survival probability data, wherein the arrhythmic sudden cardiac death survival prediction data is specific to the patient.

13. The system of claim 12, wherein the arrhythmic sudden cardiac death survival prediction data comprises an arrhythmic sudden cardiac death survival curve for the patient.

14. The system of claim 12, wherein the arrhythmic sudden cardiac death survival prediction data comprises a probability distribution representing time of predicted arrhythmic sudden cardiac death.

15. The system of claim 12, wherein the survival probability data comprises a probability distribution location datum and a probability distribution scale datum.

16. The system of claim 15, wherein the output provides a patient-specific uncertainty assessment for the arrhythmic sudden cardiac death survival prediction data based on the probability distribution scale datum.

17. The system of claim 12, further comprising a third subnetwork that combines the first survival probability data and the second survival probability data into third survival probability data, wherein the arrhythmic sudden cardiac death survival prediction data is based on the third survival probability data.

18. The system of claim 12, wherein the cardiac covariate data for the patient comprises cardiac image measurement data, risk factor data, electrocardiogram data, and medication data for the patient.

19. The system of claim 18,
wherein the cardiac image measurement data comprises at least one of left ventricle mass data or infarct size data,
wherein the risk factor data comprises ejection fraction data,
wherein the electrocardiogram data comprises at least one of heart rate data or QRS complex data, and
wherein the medication data comprises at least one of beta blocker medication data, diuretic medication data, digoxin medication data.

20. The system of claim 12, wherein the first subnetwork comprises an encoder-decoder subnetwork, and wherein the second subnetwork comprises a dense subnetwork.

21. The system of claim 12,
wherein the first subnetwork is trained with training cardiac image data, the training cardiac image data comprising times until arrhythmic sudden cardiac death events and times until non-arrhythmic-sudden-cardiac-death events, and
wherein the second subnetwork is trained with training cardiac covariate data, the training cardiac covariate data comprising times until arrhythmic sudden cardiac death events and times until non-arrhythmic-sudden-cardiac-death events.
